(19) United States
(12) Patent Application Publication
(10) Pub. No.: US 2020/0138303 A1
(43) Pub. Date: May 7, 2020

(54) SYSTEM AND METHOD FOR CUFF-LESS BLOOD PRESSURE MONITORING

(71) Applicant: The Texas A&M University System, College Station, TX (US)

(72) Inventors: Roozbeh Jafari, Austin, TX (US); Bassem Ibrahim, College Station, TX (US)

(21) Appl. No.: 16/625,226

(22) PCT Filed: Jul. 6, 2018
(86) PCT No.: PCT/US18/41100

§ 371 Date: Dec. 20, 2019

Related U.S. Application Data
(60) Provisional application No. 62/529,944, filed on Jul. 7, 2017.

(51) Int. Cl. A61B 5/021; A61B 5/053; A61B 5/00

(52) U.S. Cl. CPC A61B 5/02125; A61B 5/0535; A61B 5/681; A61B 2562/046; A61B 5/7225; A61B 5/7235; A61B 5/7221

(57) ABSTRACT

A method of non-invasive, continuous, real-time measurement of blood pressure includes applying a wearable blood-pressure monitoring device to a bodily region of a wearer. The wearable blood-pressure monitoring device includes a plurality of pulse sensors or a sensor offering a plurality of observations. The wearable blood-pressure monitoring device is calibrated such that a subset of the plurality of pulse sensors or observations are selected. The selected subset of the plurality of pulse sensors measure a pulse wave velocity and other important physiological parameters of the wearer. The measured pulse wave velocity and other important physiological parameters are converted to a blood pressure of the wearer.

### CROSS-REFERENCE TO RELATED APPLICATIONS

This patent application claims priority to, and incorporates by reference the entire disclosure of, U.S. Provisional Patent Application No. 62/529,944, filed on Jul. 7, 2017.

### BACKGROUND

### Technical Field

The present disclosure relates generally to measurement of blood pressure and more particularly, but not by way of limitation, to systems and methods for non-invasive, continuous measurement of blood pressure utilizing measurement of pulse wave velocity and other important physiological parameters.

### History of the Related Art

This section provides background information to facilitate a better understanding of the various aspects of the disclosure. It should be understood that the statements in this section of this document are to be read in this light, and not as admissions of prior art.

Hypertension is a cardiac ailment affecting a significant portion of the world's population. In order to diagnose a patient for the first time, as well as monitor the progress of a patient taking medication, it is important to measure blood pressure regularly and reliably. For repeated measurements in the long term, it is ideal for the method of measurement to be non-invasive. Moreover, the measurement device should be easy to use for a non-expert, and allow for in-home measurement, which means more frequent measurement without the need for hospital visits, and also provides the most natural context for the patient.

### SUMMARY

This summary is provided to introduce a selection of concepts that are further described below in the detailed description. This summary is not intended to identify key or essential features of the claimed subject matter, nor is it to be used as an aid in limiting the scope of the claimed subject matter.

Various aspects of the disclosure relate to a method of non-invasive, continuous, real-time measurement of blood pressure. The method includes receiving a plurality of pulse observations from a plurality of sensors disposed on a blood-pressure monitoring device. The blood-pressure monitoring device is arranged on a bodily region of a wearer. A subset of the plurality of pulse observations received from a subset of the plurality of sensors is selected. A pulse wave velocity and other important physiological parameters of the wearer is measured via the selected subset of the plurality of pulse observations. The measured pulse wave velocity and other important physiological parameters are converted to a blood pressure of the wearer.

Various aspects of the disclosure relate to a wearable blood-pressure monitoring device. The wearable blood-pressure monitoring device includes a controller. A plurality of pairs of electrodes are arranged to be in contact with a bodily region of a wearer. A plurality of pairs of sensors are coupled to the controller. Each sensor of the plurality of pairs of sensors is positioned in contact with the bodily region of the wearer and interposed between a pair of electrodes of the plurality of pairs of electrodes. A securement device is coupled to the controller.

Various aspects of the disclosure relate to a method of calibrating a wearable blood-pressure monitoring device. The method includes receiving a plurality of pulse observations from a plurality of sensors disposed on a blood-pressure monitoring device. The blood-pressure monitoring device is arranged on a bodily region of a wearer. Signals from different combinations of sensors of the plurality of sensors are captured. A subset of sensors of the plurality of sensors that provide actionable data for measurement of pulse wave velocity and other important physiological parameters are determined. Pulse wave velocity is measured as a phase shift between the subset of sensors of the plurality of sensors, while the other important physiological parameters are measured from the pulse rate, amplitude, and time duration of readings of the sensors.

### BRIEF DESCRIPTION OF THE DRAWINGS

The disclosure is best understood from the following detailed description when read with the accompanying figures. It is emphasized that, in accordance with standard practice in the industry, various features are not drawn to scale. In fact, the dimensions of various features may be arbitrarily increased or reduced for clarity of discussion.

FIG. 1A is a schematic diagram of an illustrative wearable blood-pressure monitoring device showing placement on a wearer's wrist;

FIG. 1B is a photographic illustration of the illustrative wearable blood-pressure monitoring device of FIG. 1A;

FIG. 1C is a photographic illustration of the illustrative wearable blood-pressure monitoring device of FIG. 1A showing an underside thereof;

FIG. 2 is a flow diagram illustrating an illustrative process for cuff-less measurement of blood pressure;

FIG. 3 is a block diagram of an algorithm for blood-pressure measurement utilizing a wrist worn bio-impedance sensor array;

FIG. 4 is a block diagram of an illustrative blood-pressure monitoring device; and

FIG. 5 is a plot of a bio-impedance signal according to aspects of the disclosure.

### DETAILED DESCRIPTION

Various embodiments of the present disclosure will now be described more fully with reference to the accompanying drawings. The disclosure may, however, be embodied in many different forms and should not be construed as limited to the embodiments set forth herein.

According to current estimates, there are approximately 30 million people in the United States who suffer from cardiovascular disease. This incidence of cardiovascular disease is attributable to approximately $555 billion in health care costs and lost productivity. According to predictions from the American Heart Association, the costs will rise to $1.1 trillion by 2035. Blood pressure monitoring is well-recognized as being important in the diagnosis and management of cardiovascular disease.

Currently, blood pressure measurement involves the use of a sphygmomanometer, which has an inflatable cuff. Measurement with a sphygmomanometer allows wearers to autonomously measure blood pressure at home. However, the sphygmomanometer can be considered invasive, given the inflated pressure that affects the blood flow and causes discomfort. This discomfort could lead to reluctance on the part of the patient to take readings, which in turn leads to less data for the doctor to use and an increased likelihood of missed diagnoses of critical conditions. Furthermore, use of the sphygmomanometer provides a one-time measurement while the wearer is seated expressly for the purpose of measuring blood pressure. In other words, the sphygmomanometer does not provide a continuous data stream that is paired with contextual information such as, for example, the activity being performed at the time of informative blood-pressure trends. For example, it would be insightful to know if the daily commute by car and a road disturbance are causing the patient's blood pressure to spike up significantly.

It has previously been shown that pulse wave velocity pulse transit time is correlated with blood pressure, so pulse wave velocity or pulse transit time could be translated to blood pressure given effective calibration techniques. However, most existing solutions do not represent a single integrated pulse transit time measurement device in a wearable form factor that is easy to use. Pulse transit time is defined as the time taken by a pressure pulse to travel through a vessel between two fixed points during each cardiac cycle.

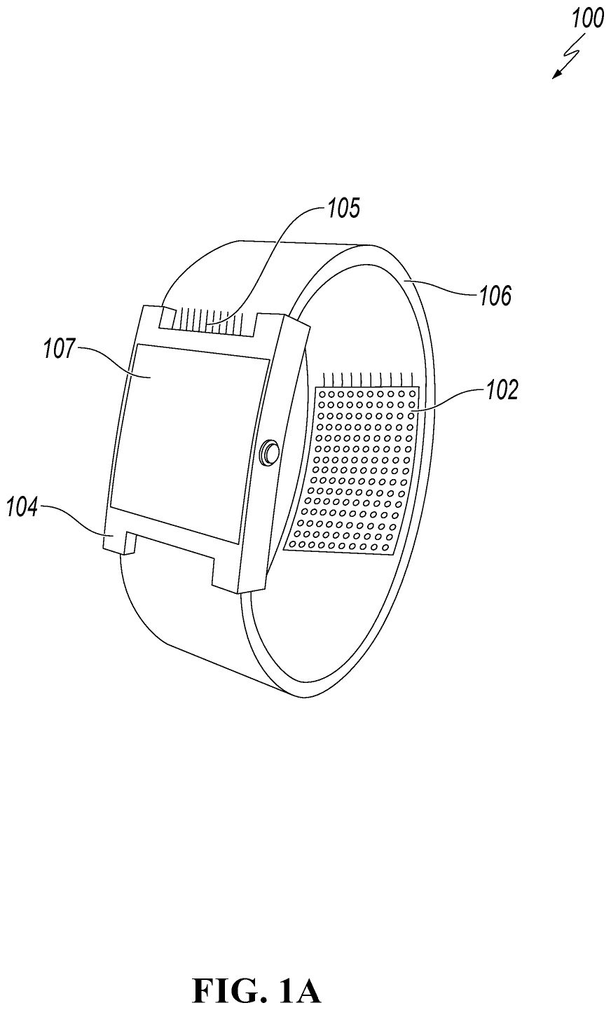

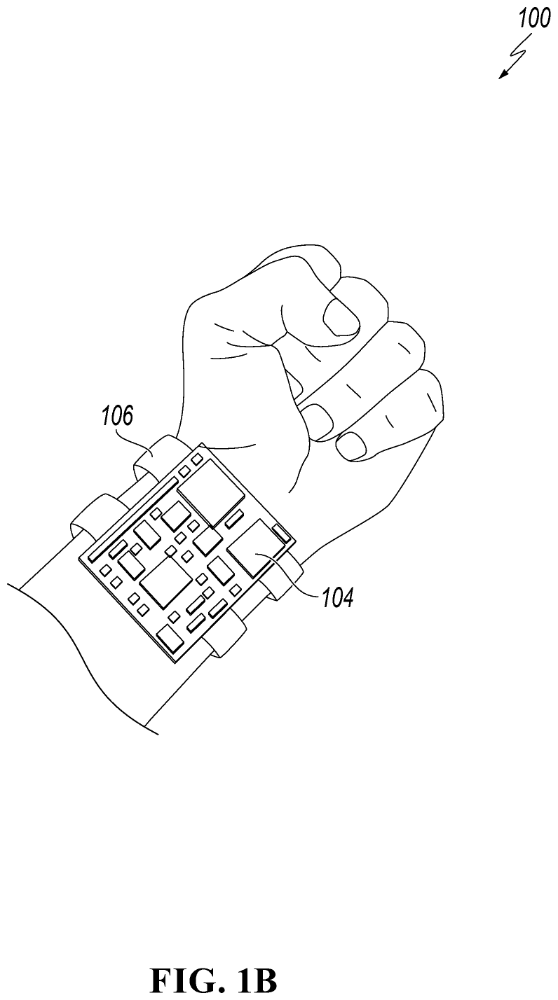

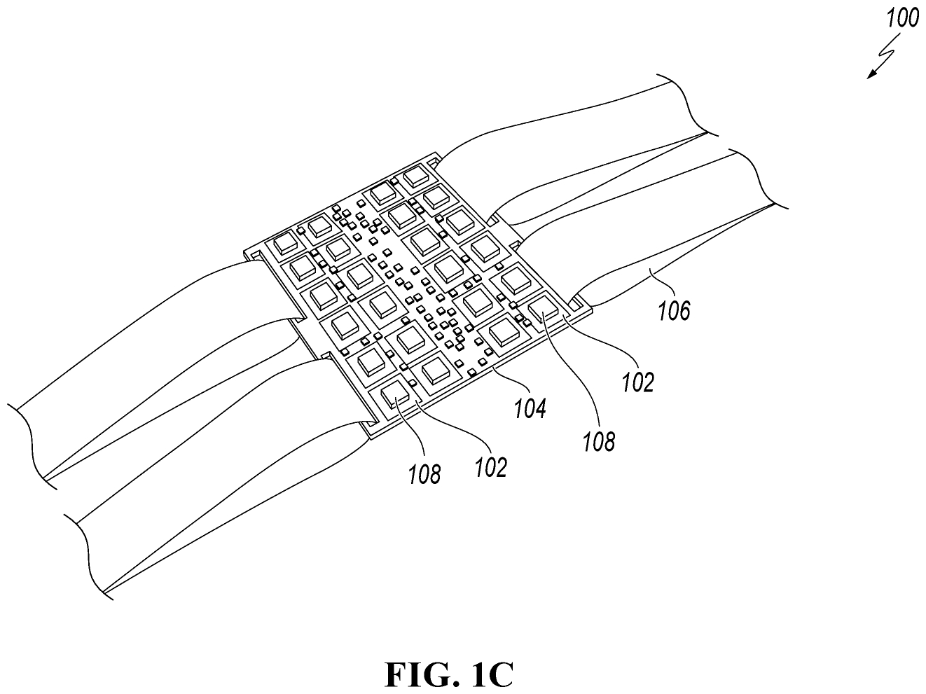

FIG. 1A is a perspective view of an illustrative wearable blood-pressure monitoring device 100. The wearable blood-pressure monitoring device 100 includes a plurality of pulse sensors 102 that are coupled to a controller 104 via an interconnection 105. In a typical embodiment, the wearable blood-pressure monitoring device 100 may include a display 107. In other embodiments, the display may be remote such as, for example, on a smart phone display or on a remote health-care provider monitor. In other embodiments, the observations may be stored in a storage device for further analysis. In various embodiments, the wearable blood-pressure monitoring device 100 includes, for example, straps 106 that facilitate securement of the wearable blood-pressure monitoring device 100 to a bodily region of the wearer. In various embodiments, however, the wearable blood-pressure monitoring device 100 could also be contained in, for example, a patch constructed with a bio-compatible adhesive that facilitates securement of the wearable blood-pressure monitoring device 100 to the bodily region of the wearer. The plurality of pulse sensors 102 are arranged in an array on, for example, an inside aspect of a wrist region of the wearer. The plurality of pulse sensors 102 are, in various embodiments, arranged in an array such as, for example, a 3×3 array. In various embodiments, utilization of an array of pulse sensors 102 provides redundancy in the event of failure of one or more of the pulse sensors 102 or sub-optimal placement of one or more of the sensors 102. In various embodiments, the plurality of pulse sensors 102 could include a bio-impedance sensor, a bio-potential sensor, an optical sensor such as, for example, a photoplethysmogram (PPG) sensor, or other types of physiological sensor or a combination of them. The interconnection 105 couples the controller 104 to the plurality of pulse sensors 102. In various embodiments, the interconnection 105 is a wired connection such as, for example, through the strap 106; however, in other embodiments, a wireless connection could be utilized. The controller 104 has the capability to communicate with other devices such as, for example, a cellular telephone or a cloud computing device via, for example, a cellular network or a wireless protocol.

Photoplethysmogram (“PPG”) is an optical measurement that characterizes a volume flow of blood through a measurement site. A PPG sensor comprises a LED paired with a photodiode placed externally on the surface of the wearer's skin. The LED emits light of suitable wavelength—typically red, infrared, or green—into the skin and the photodiode transduces the reflected light into a proportionate amount of current. As blood rushes into the blood vessels beneath the measurement site during each cardiac cycle, the blood absorbs a certain amount of the incident light, which in turn means there is a sharp decrease in the light reflected back to the photodiode. The amount of reflection then gradually increases before falling again at the time of the next pulse. Thus, the arrival time of a new pulse at the measurement site over time can be inferred.

Bio-impedance is the electrical impedance of body tissues to an applied current, which can be used to measure the amounts of body fluids such as, for example, blood volume in blood vessels. The bio-impedance variations (AZ) correspond to variations of blood volume due to the heart's activity in each cardiac cycle. Bio-impedance can be measured by applying, for example, an AC sinusoidal current through a pair of electrodes and sensing the voltage with two different electrodes near the excitation electrode. The measured voltage is a sinusoidal voltage at the same frequency as the applied current and its amplitude is correlated with bio-impedance. Signal transformation is used to extract bio-impedance by multiplying the sensed signal by the excitation signal and current.

FIG. 1B is a photographic illustration of the illustrative wearable blood-pressure monitoring device 100. FIG. 1C is a photographic illustration of the illustrative wearable blood-pressure monitoring device of 100 showing an underside thereof. Referring to FIGS. 1B and 1C collectively, the straps 106 secure the wearable blood-pressure monitoring device 100 to a bodily region of the wearer such as, for example, on an interior of the wearer's wrist. In various embodiments, the wearable blood-pressure monitoring device 100 could be disposed in other locations on the wearer's body including, for example, an upper arm region, a neck region, a head region, or a chest region of the wearer. In FIG. 1C, the plurality of pulse sensors 102 is shown. By way of example, the plurality of pulse sensors 102 are illustrated in FIGS. 1B-1C as being bio-impedance sensors; however one skilled in the art will recognize that other types of sensors including, for example, optical sensors and bio-potential sensors could be utilized alone or in combination with the bio-impedance sensors. As shown in FIG. 1C, in the case of bio-impedance sensors, each pulse sensor of the plurality of pulse sensors 102 includes an electrode 108 that facilitates transmission of the applied electrical current to the wearer's skin. The electrode 108 is constructed of an electrically-conductive non-corrosive biocompatible material.

During operation, the wearable blood-pressure monitoring device 100 is applied to the bodily region of the wearer. The controller 104 receives input from the plurality of pulse sensors 102 regarding underlying vasculature and cardiac phenomena. The controller 104 calibrates the wearable blood-pressure monitoring device 100. As used herein, “calibration” refers to a determination, by the controller 104, of a subset of pulse sensors of the plurality of pulse sensors 102 that will provide actionable data for measurement of pulse transit time and pulse wave velocity. In various embodiments, the subset of pulse sensors is a pair of pulse sensors of the plurality of pulse sensors 102. This calibration is based on the view of sensors of the underlying cardiac and vasculature phenomena and a quality measurement of each sensor of the plurality of sensors at various layers of the bodily region. For example, in various embodiments, the calibration is based at least in part on the type of sensor utilized as well as a location of the pulse sensors of the plurality of pulse sensors 102 relative to, for example, a blood vessel. In various embodiments, pulse sensors 102 not included in the selected subset may be, for example, deactivated; however, in other embodiments, pulse sensors 102 not included in the selected subset are simply disregarded by the controller 104. In this manner, utilization of the array of pulse sensors 102 provides redundancy in the event of failure of one or more of the pulse sensors 102 or sub-optimal placement of one or more of the sensors 102.

During operation, when the wearable blood-pressure monitoring device 100 is activated, the wearable blood-pressure monitoring device 100 enters a calibration mode. In the calibration mode, different combinations of sensors of the plurality of pulse sensors 102 are activated and signals are captured for approximately two to approximately three heartbeats. Switching between the different combinations of sensors of the plurality of pulse sensors 102 occurs quickly so that the bio-impedance is captured for an exhaustive set of sensors. Due to large blood flow in the arteries, the peak-to-peak amplitude of the bio-impedance sensed by pulse sensors in close proximity to a blood vessel such as, for example, the radial artery or the ulnar artery, will be higher. Once a pair of sensors of the plurality of sensors 102 that exhibits the best contact quality and location is determined, the pulse wave velocity is measured as a phase shift between the two bio-impedance signals divided by the distance between the selected sensors. The other important physiological parameters and the contact quality are simultaneously estimated and stored along with the bio-impedance and pulse wave velocity measurements. For example, one indicator of signal-quality degeneration is a reduced amplitude of a sensed signal.

In various embodiments, the controller 104 may also consider, for example, a contact quality between a pulse sensor of the plurality of pulse sensors 102 and the wearer's skin and select a suitable set of pulse sensors for observation. The plurality of pulse sensors 102 may be equipped with the capability to monitor the contact quality with the skin as the wearer performs daily physical activities, which may result in movement or misplacement of one or more pulse sensors of the plurality of pulse sensors 102. Such movement of the plurality of pulse sensors 102 may result in certain pulse sensors of the plurality of pulse sensors 102 not having suitable contact with the wearer's skin. For example, if the wearer's skin is not suitable for bio-impendence electrodes due to, for example, poor contact or accumulation of debris, the controller 104 could sense poor signal quality from the bio-impedance sensor and select, for example, an optical sensor over a bio-impedance sensor. In various embodiments, the contact quality determination can be accomplished by reduced signal amplitude or other advanced techniques such as the introduction of an out-of-band common mode voltage and measuring common mode rejection ratio (“CMRR”) due to degrading contact quality and contact impedance. In other situations, the controller 104 could select, for example, two pulse sensors located closest to a blood vessel such as, for example, an artery, a vein, a capillary, or other vessels. In various embodiments, the blood vessel may be, for example, a radial artery and/or an ulnar artery or other important underlying tissue. In this manner, the wearable blood-pressure monitoring device 100 may, in various embodiments, combine the modalities of bio-impedance sensing and, for example, photoplethysmography.

Calibration of the wearable blood-pressure monitoring device 100 may, in various embodiments, be repeated periodically during use to account for factors including, changes of bodily position of the wearer and changes of position of the wearable blood-pressure monitoring device 100 relative to the wearer's body. In this manner, the subset of the plurality of pulse sensors 102 is selected based on a location of the subset of the plurality of pulse sensors 102 on the wearer's body. In various embodiments, the wearable blood-pressure monitoring device 100 may include a single sensor in lieu of the plurality of pulse sensors 102. Such a single sensor may offer a plurality of observations. For example, a vision sensor may be utilized as a single sensor but, in various embodiments, could incorporate pixels or individual sensing points and provide a plurality of sensor observations via an array of pixels or sensing points.

Still referring to FIGS. 1A-1C, during operation, the plurality of pulse sensors 102 provide multiple views of the vasculature and cardiac phenomenon underlying the bodily location of the wearer. For example, the controller 104 may determine, based at least in part on, for example, a signal amplitude received from the plurality of pulse sensors 102 that at least one sensor of the plurality of pulse sensors 102 is positioned near a blood vessel such as, for example, an artery, a vein, a capillary, or other vessels. In various embodiments, the blood vessel may be, for example, the radial artery or the ulnar artery or other important underlying tissues. The controller 104 adjusts the frequency and the amplitude of the current applied to the wearer in an effort to control the coverage area of the plurality of pulse sensors 102. In this manner, certain parameters of the pulse sensors could be varied including, but not limited to changing a frequency of excitation current in bio-impedance sensors, performing a frequency sweep, or changing a current level injected into optical sensors, changing the wavelength of optical excitation to adjust the coverage area or by observing other related physiological phenomena. In various embodiments, such functionality may also improve the sensitivity of detection and provide zoom capabilities. The controller 104 recognizes various postures of the wearer including, for example differing positions of the wearer's arm. Responsive to a determination that the wearer's position has changed, the controller 104 may, in various embodiments, recalibrate the plurality of pulse sensors 102.

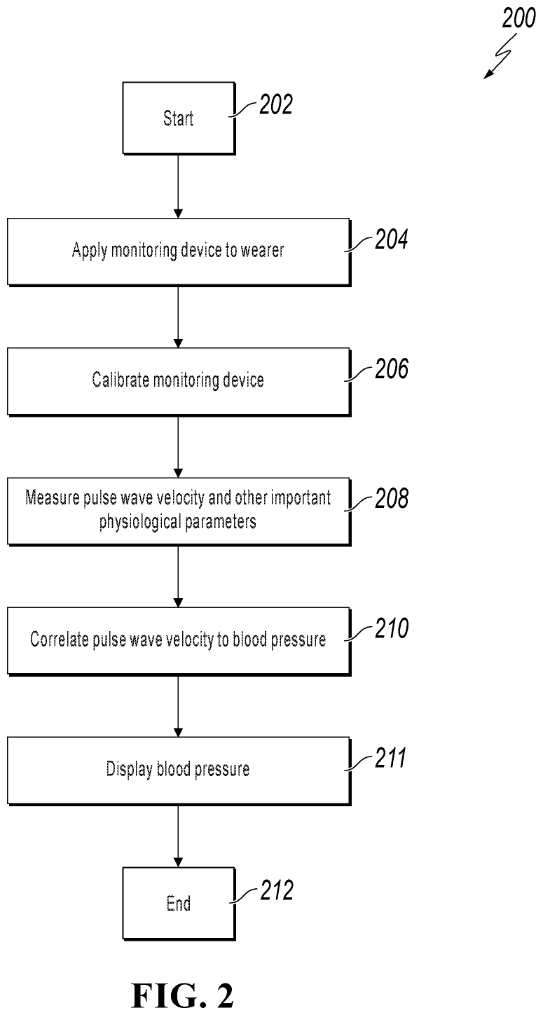

FIG. 2 is a flow diagram illustrating an illustrative process 200 for cuff-less measurement of blood pressure. The process 200 begins at block 202. At block 204, the wearable blood-pressure monitoring device 100 is applied to a bodily region of a wearer. At block 206, the wearable blood-pressure monitoring device 100 is calibrated such that a subset of the plurality of pulse sensors 102 are utilized. In embodiments utilizing a single sensor, that provides a plurality of observations, suitable observations are selected. At block 208, the subset of the plurality of pulse sensors 102 are utilized to measure pulse transit time, pulse wave velocity, and other important physiological parameters. At block 210, the measured pulse transit time and pulse wave velocity, along with the other important physiological parameters are converted to blood pressure leveraging signal processing, statistical or machine learning techniques taking into account, for example, the wearer's posture. At block 211, the controller 104 displays the wearer's blood pressure information. In various embodiments, the conversion of pulse wave velocity and other important physiological parameters may be by way of, for example, a lookup table, regression techniques, or other methodology. In this manner, the wearable blood-pressure monitoring device 100 provides a real-time, continuous, and non-invasive measurement of blood pressure. In various embodiments, measurements from the plurality of pulse sensors 102 may be stored to allow calculation and display of the wearer's blood pressure at a later time. The process 200 ends at block 212.

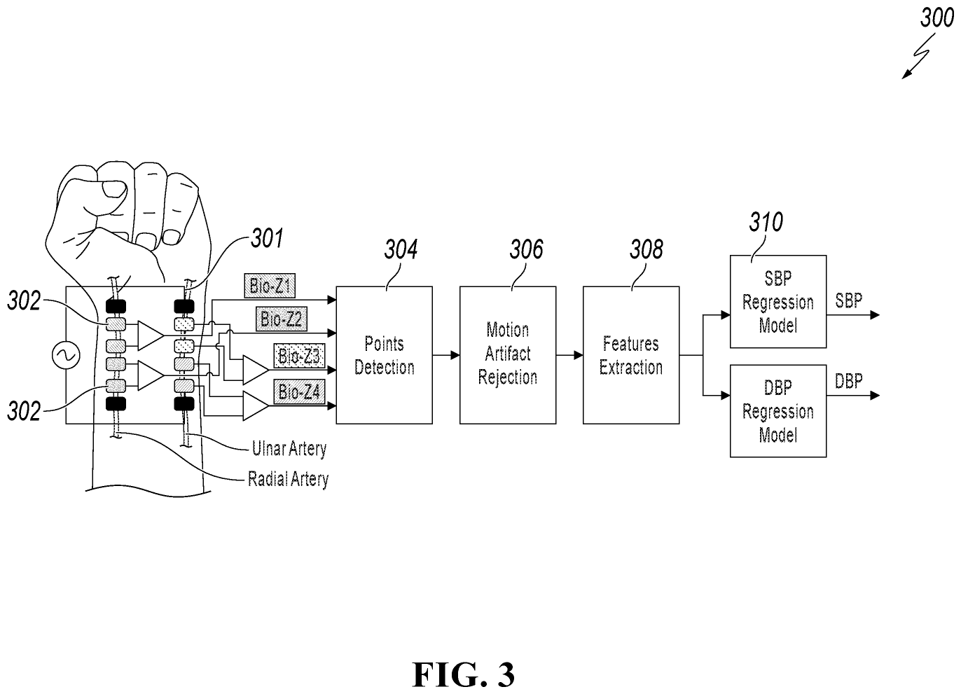

FIG. 3 is a block diagram of an algorithm for blood-pressure measurement utilizing a blood-pressure monitoring device 300. By way of example, the sensor array 301 is located on a wrist of a wearer. The sensor array includes a plurality of sensors 302. In various embodiments, the sensors 302 are, for example, bio-impedance sensors, optical sensors, or any other appropriate type of sensor. The sensors 302 are arranged proximate to a blood vessel such as, for example, an artery, a vein, a capillary, or other vessels. In various embodiments, the blood vessel could be, for example, a radial artery and/or an ulnar artery of the wearer or other important underlying tissues. At step 304, the controller 104 detects the pairs of sensors 302 that are in proximity to a blood vessel, which, in various embodiments, may be, for example, the radial artery and/or the ulnar artery or other important underlying tissues. At step 306, the controller rejects artifacts of motion related to movements through various algorithms such as, for example, Kalman filters, particle filters, or others. Motion artifact rejection techniques may also leverage additional sensors including motion sensors such as, for example, accelerometers, gyroscopes, and magnetometers. At step 308, the controller 104 utilizes the sensors 302 to extract features from the vasculature underlying the wearer's wrist. At step 310, the controller utilizes a regression model to determine systolic blood pressure and diastolic blood pressure.

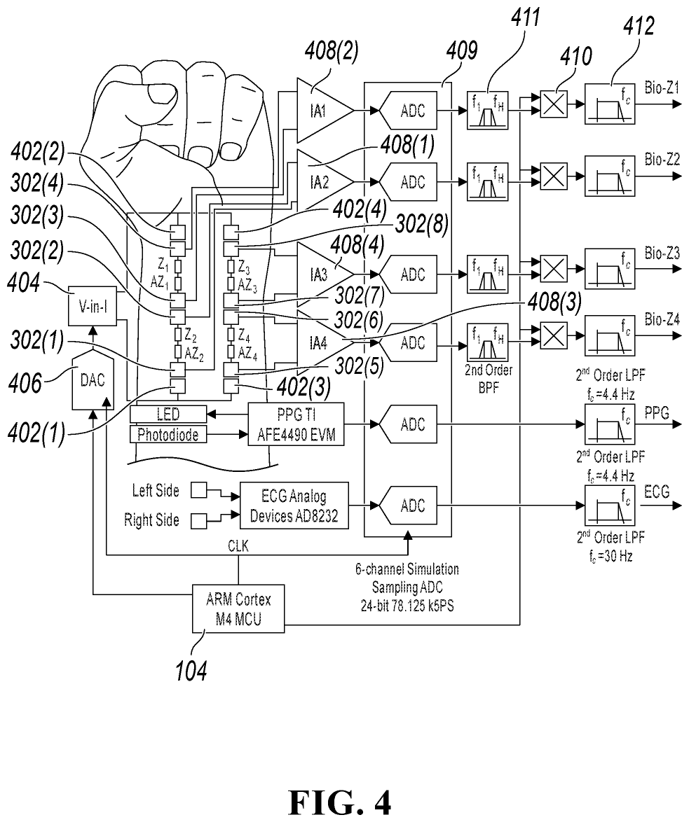

FIG. 4 is a block diagram of an embodiment of the blood-pressure monitoring device 300. A plurality of electrodes 402(1)-402(4) are placed on the wrist of the wearer. In various embodiments, two electrodes 402(1) and 402(2) are arranged, for example, on along the radial artery and two electrodes 402(3) and 402(4) are arranged, for example, along the ulnar artery. The plurality of electrodes are electrically coupled to a voltage-to-current source 404. The voltage-to-current source 404 is electrically coupled to a digital-to-analog converter 406, which is electrically coupled to the controller 104. In a typical embodiment, the digital-to-analog converter 406 facilitates modulation of frequency and voltage that is supplied to the electrodes 402.

Still referring to FIG. 4, the sensors 302(1), 302(2), 302(3), and 302(4) are arranged in pairs along the radial artery between the electrodes 402(1) and 402(2) and the sensors 302(5), 302(6), 302(7), and 302(8) are arranged in pairs along the ulnar artery between the electrodes 402(3) and 402(4). Along the radial artery, bio-impedance is measured between the sensor 302(1) and the sensor 302(2). Bio-impedance is also measured between the sensor 302(3) and the sensor 302(4). Pulse transit time is measured between the pair of sensors 302(1)-302(2) and the pair of sensors 302(3)-302(4). Along the ulnar artery, bio-impedance is measured between the sensor 302(5) and the sensor 302(6). Bio-impedance is also measured between the sensor 302(7) and the sensor 302(8). Pulse transit time is measured between the pair of sensors 302(5)-302(6) and the pair of sensors 302(7)-302(8). By utilizing the sensors 302(1)-302(8), redundancy is provided in the event of failure of one or more of the sensors 302(1)-302(8). Additional important physiological parameters are measured from the aforementioned pairs of sensors including 302(1)-302(2), 302(3)-302(4), 302(5)-302(6), and 302(7)-302(8).

Still referring to FIG. 4, the sensor 302(1) and the sensor 302(2) are coupled to an amplifier 408(1). The sensor 302(3) and the sensor 302(4) are coupled to an amplifier 408(2). The sensor 302(5) and the sensor 302(6) are coupled to an amplifier 408(3) and the sensor 302(7) and the sensor 302(8) are coupled to an amplifier 408(4). The amplifiers 406(1)-(4) are electrically coupled to an analog-to-digital converter 409. The analog-to-digital converter 409 is electrically coupled to a band-pass filter 411. The band-pass filter 411 is electrically coupled to a de-modulator 410. The de-modulator 410 is further electrically coupled to a low-pass filter 412. The signals that are correlated to bio-impedance are then passed to the controller 104 for further processing and conversion to bio-impedance. By way of example, the electrodes 402(1)-402(4) and the sensors 302(1)-302(8) are described in FIG. 4 as being positioned along a radial artery and an ulnar artery of the wearer. In other embodiments, the electrodes 402(1)-402(4) and the sensors 302(2)-302(8) may be positioned relative to any blood vessel such as, for example, an artery, a vein, a capillary, or other vessel.

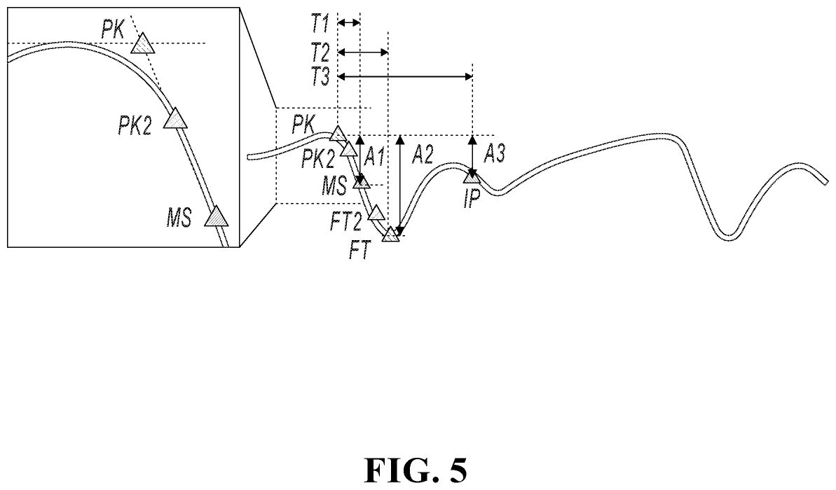

FIG. 5 is a plot of a bio-impedance signal. For each bio-impedance signal, six characteristic points are detected for every beat. Every heart beat, the bio-impedance signal decreases from a peak (PK) to a foot (FT), which indicates a sudden increase in the blood volume due to the arrival of the pressure pulse. The bio-impedance peak (PK) represents the diastolic phase while the bio-impedance foot (FT) represents the systolic phase of the heart beat. The decreasing slope section is abstracted by five points as shown in FIG. 5. The bio-impedance peak (PK) and the bio-impedance foot (FT) are detected by the intersection of the tangent to the slope with the horizontal line from the maximum and the minimum of the signal, respectively. In addition, the points of maximum change in the slope at the peak (PK2) and the foot (FT2) of the signal are detected. The maximum slope (MS) point is also an important point in the middle of the decreasing slope section. The sixth point is detected from the inflection point (IP), which results from the reflection of the pressure pulse. All these points are identified using zero crossing, peak and foot points of the first and the second derivative of the Bio-Z signal. All of these points are identified using zero crossing, peak and foot points of the first and the second derivative of the bio-impedance signal.

Still referring to FIG. 5, the bio-impedance signals are used to generate 90 features for each heart beat that can model the pulse transit time on a blood vessel such as, for example, the radial artery and the ulnar artery. These features represent important physiological parameters including pulse transit time and other parameters. In addition, the mean features are generated by taking the average of beat-by-beat features over every 12 beats with 50% overlap. The mean features provide better results because the mean features remove high-frequency variations within around 10 seconds while the BP can be assumed constant. In various embodiments, the mean features may include:

Pulse Transit Time Features: The pulse transit time features are the time delay between each pair of bio-impedance signals measured at the points PK, PK2, MS, FT2, and FT (30 features);

Heart Rate Features: The heart rate features are the time interval between two successive beats for each bio-impedance signal measured at the points PK, PK2, MS, FT2, and FT (20 features);

Time Features: These features are the time interval from the PK to the MS, FT and IP points, which are T1, T2, and T3 respectively as shown in FIG. 5 (12 features);

Amplitude Features: These are the difference in amplitude from PK to MS points (A1) and from PK to FT points (A2) in addition to the ratio of the amplitude of the MS point (A1) to the amplitude of the IP point (A3) (12 features); and

Area Ratio Features: These features are the areas under the bio-impedance curve between the PK, MS, FT and IP points which represent the total peripheral resistance (16 features).

Various embodiments of the disclosure may relate to a wearable blood-pressure monitoring device. The wearable blood-pressure monitoring device includes a means for controlling. In various embodiments, the means for controlling may be, for example, a microprocessor, a micro control unit, a computer numeric controller, a proportional/derivative/integral controller, or a machine control unit such as, for example, a Cortex-M4 controller produced by Arm Holdings. The wearable blood-pressure monitoring device also includes means for sensing. The means for sensing are positioned to be in contact with a bodily region of the wearer. In various embodiments, the means for sensing may include bio-impedance sensors, optical sensors, acoustic sensors, or any other type of sensors capable of observing cardiac phenomena. A means for securing is coupled to the means for controlling. In various embodiments, the means for securing may include, for example, a strap, a wrist band, or a bio-compatible adhesive.

Various embodiments of the invention relate to a computer-program product that includes a non-transitory computer-usable medium having computer-readable program code embedded therein. The computer-readable program code may be executed to implement a method that includes receiving a plurality of pulse observations from a plurality of sensors disposed on a blood-pressure monitoring device, selecting a subset of the plurality of pulse observations, and measuring, via the selected subset of the plurality of pulse observations, a pulse wave velocity and other important physiological parameters of a wearer. The measured pulse wave velocity and other important physiological parameters are then converted to a blood pressure of the wearer.

A particular advantage provided by at least one of the disclosed embodiments includes accurate and precise measurement of blood pressure in a non-invasive and continuous manner. For example, the controller 104 of the wearable blood-pressure monitoring device 100 may be configured, for example, to dynamically select a subset of a plurality of pulse observations received from a plurality of sensors. The selection of the subset of the plurality of pulse observations may be based, at least in part, on indicia of an accuracy and a precision of the pulse observations. In this manner, an accuracy and a precision of a pulse wave velocity determined based on the subset of the plurality of pulse observations may be greater than an accuracy and a precision of a pulse wave velocity determined through conventional techniques. Consequently, a blood pressure value determined based on the pulse wave velocity generated from the subset of the plurality of pulse observations may correspond to a more accurate and a more precise blood-pressure measurement generated through or derived from conventional techniques.

The disclosure may enhance the operation of a controller by reducing an amount of received data upon which measurement of pulse transit time and calculation of pulse wave velocity are performed. In particular, rather than measuring pulse transit time and calculating pulse wave velocity on data received from all sensors of a wearable blood-pressure monitoring device or on all data received from any sensor of the wearable blood-pressure monitoring device, the controller may dynamically select actionable data (i.e., a subset of all data received) upon which the pulse transit time can be measured and upon which pulse wave velocity can be calculated. In this manner, the controller may dynamically ignore non-actionable data, such as data that may generate imprecise or inaccurate measurements of pulse transmit time and imprecise or inaccurate calculations of pulse wave velocity. In addition to enhancing an accuracy and a precision of a value of a blood pressure corresponding to the pulse wave velocity, the disclosure may reduce power consumed by the controller, since the controller is not performing measurements of pulse transmit time and calculations of pulse wave velocity on all data received from any sensor. Further, the disclosure may enhance a speed with which the controller may perform the pulse velocity calculations, since the controller may perform the pulse velocity calculations on actionable data rather than on all data received from any sensor. In this manner, the disclosure may enhance the performance of the controller.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions and alterations herein without departing from the spirit and scope of the disclosure. The scope of the disclosure should be determined by the language of the claims that follow. The term “comprising” within the claims is intended to mean “including at least” such that the recited list of elements in a claim are an open group. The terms “a,” “an,” and other singular terms are intended to include the plural forms thereof unless specifically excluded.

What is claimed is:

1. A method of non-invasive, continuous, real-time measurement of blood pressure, the method comprising:
receiving a plurality of pulse observations from a plurality of sensors disposed on a blood-pressure monitoring device, the blood-pressure monitoring device being arranged on a bodily region of a wearer;
selecting a subset of the plurality of pulse observations received from a subset of the plurality of sensors;
measuring, via the selected subset of the plurality of pulse observations, a pulse wave velocity and other important physiological parameters of the wearer; and
converting the measured pulse wave velocity and other important physiological parameters to a blood pressure of the wearer.

2. The method of claim 1, wherein the selecting is based, at least in part, on a signal quality of each sensor of the plurality of sensors.

3. The method of claim 1, further comprising providing, via the plurality of sensors, data corresponding to multiple views of vasculature and cardiac phenomena underlying the bodily region of the wearer.

4. The method of claim 3, wherein a signal quality is determined at least in part by a location of a sensor of the plurality of sensors relative to a blood vessel.

5. The method of claim 1, wherein the subset comprises at least two pairs of sensors of the plurality of sensors that facilitates improvement of at least one of accuracy and redundancy.

6. The method of claim 1, further comprising measuring a pulse transit time and other important physiological parameters between the sensors of the subset of the plurality of sensors.

7. The method of claim 1, wherein the plurality of sensors comprise bio-impedance sensors, optical sensors, other physiological sensors that can measure pulse arrival time or other important physiological parameters, or a combination thereof.

8. The method of claim 1, further comprising adjusting parameters of the plurality of sensors to control a sensing coverage area of the plurality of sensors or observations.

9. The method of claim 1, further comprising detecting a quality of a signal or a quality of contact between the sensor and a wearer's skin sensed by the plurality of sensors.

10. The method of claim 1, further comprising detecting a posture of the wearer or a location of the body region where the sensor is placed in reference to the wearer's body.

11. The method of claim 10, further comprising calibrating the pulse wave velocity detection algorithm responsive to the view of vasculature and cardiac phenomena and the detected posture.

12. The method of claim 1, wherein the plurality of sensors provide redundancy.

13. A wearable blood-pressure monitoring device comprising:
a controller;
a plurality of pairs of electrodes arranged to be in contact with a bodily region of a wearer;
a plurality of pairs of sensors coupled to the controller, each sensor of the plurality of pairs of sensors being positioned in contact with the bodily region of the wearer and interposed between a pair of electrodes of the plurality of pairs of electrodes; and
a securement device coupled to the controller.

14. The wearable blood-pressure monitoring device of claim 13, wherein the securement device is a strap or other mechanism configured to facilitate coupling of the plurality of sensors to the bodily region.

15. The wearable blood-pressure monitoring device of claim 13, wherein the plurality of sensors comprises a bio-impedance sensor, an optical sensor, or other types of sensors that can measure pulse arrival time or other important physiological parameters.

16. The wearable blood-pressure monitoring device of claim 13, further comprising a display or storage device coupled to the controller.

17. The wearable blood-pressure monitoring device of claim 13, wherein the plurality of sensors are arranged in an array or other geometric forms.

18. A method of calibrating a wearable blood-pressure monitoring device, the method comprising:
receiving a plurality of pulse observations from a plurality of sensors disposed on a blood-pressure monitoring device, the blood-pressure monitoring device being arranged on a bodily region of a wearer;
capturing signals from different combinations of sensors of the plurality of sensors;
determining a subset of sensors of the plurality of sensors that provide data actionable for a determination of pulse wave velocity and other important physiological parameters; and
measuring pulse wave velocity as a phase shift between the subset of sensors of the plurality of sensors.

19. The method of claim 18, wherein the determining comprises evaluating at least one of signal strength and a location of a sensor of the plurality of sensors.

20. The method of claim 19, wherein the determining further comprises measuring peak-to-peak amplitude of bio-impedance.
